(19) United States
(12) Patent Application Publication
(10) Pub. No.: US 2007/0196282 A1
(43) Pub. Date: Aug. 23, 2007

(54) Medical diagnostic ultrasound with temperature-dependent contrast agents

(75) Inventor: Nelson H. Oliver, Sunnyvale, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/359,272

(22) Filed: Feb. 21, 2006

(51) Int. Cl. A61K 49/22

(52) U.S. Cl. 424/9.52

(57) ABSTRACT

A temperature characteristic is detected with medical diagnostic ultrasound. Microbubble contrast agents have a phase change near 37 degrees Celsius. The phase change alters the pressure required to destroy the contrast agent or cause absorption of the contrast agent. Since the contrast agents are sensitive to local temperature, ultrasound may identify locations of elevated temperature, such as associated with inflammation.

### BACKGROUND

The present embodiments relate to medical diagnostic ultrasound. In particular, ultrasound signals indicate temperature information for a scanned region.

Inflammation is found in many disease processes. Determining temperatures in a patient may allow identification of the disease process. However, determining the temperatures at particular spatial locations within a patient may be difficult.

Catheter based sensors have been proposed for identifying vulnerable plaques by their elevated temperature. A temperature probe is inserted into an artery of the patient to determine the temperature. By positioning the temperature probe in different locations, the location of vulnerable plaque may be identified. However, inserting the probe within a patient may create increased risk to the patient and/or may trigger rupture of the very plaques that are to be treated. The partial occlusion of the vessel and the thermal mass of the catheter may corrupt the temperature measurements.

Noninvasive sensing has also been proposed. For example, infrared thermography may show relative temperatures within a patient. As another example, MRI may noninvasively image internal patient temperature. However, both of these imaging types may be expensive or otherwise undesired.

In U.S. Pat. No. 6,368,275, Method and Apparatus for Diagnostic Medical Information Gathering, Hyperthermia Treatment, or Directed Gene Therapy, micro-instruments are used for ultrasound imaging. A micro-instrument suitable for property imaging in a body is less than one millimeter in each dimension. The micro-instrument includes a temporarily deformable lid or cantilever. An ultrasonically observable property of the micro-instrument varies as a function of a physiological property of the body, such as temperature. However, micro-instruments may be difficult to manufacture and/or may be difficult for the liver to remove from the blood stream.

Another physiological property has been measured using ultrasound. Contrast agents, such as microbubbles, are injected into a person. The response of the contrast agents is measured to determine local amounts of pressure within the patient. However, some disease processes may not generate pressure differences.

### BRIEF SUMMARY

By way of introduction, the preferred embodiments described below include methods, systems, contrast agents and computer readable media for detecting a temperature characteristic with medical diagnostic ultrasound. Microbubble contrast agents have a phase change near 37 degrees Celsius. The phase change alters the pressure required to destroy the contrast agent or cause absorption of the contrast agent. Since the contrast agents are sensitive to local temperature, ultrasound may identify locations of elevated temperature, such as associated with inflammation, based on the contrast agent response to acoustic energy.

In a first aspect, a method is provided for detecting a temperature characteristic with a medical diagnostic ultrasound system. A plurality of microbubbles along at least a scan line is insonified. The temperature characteristic is determined along at least a portion of the scan line as a function of a response to the insonifying.

In a second aspect, a computer readable storage medium has stored therein data representing instructions executable by a programmed processor for detecting a temperature characteristic with a medical diagnostic ultrasound system. The instructions are for transmitting, sequentially, acoustic energy at different intensities into a region, receiving signals responsive to the acoustic energy and contrast agents operable to change state as a function of temperature, the signals associated with the region, and determining a relative temperature as a function of the signals.

In a third aspect, contrast agents comprise microbubbles for in vivo imaging with ultrasound. The contrast agents are destroyable or absorbable in response to different levels of acoustic energy. A lipid material has a melting characteristic within a range of temperatures from temperatures associated with inflammation of biological tissue to temperatures associated with non-inflamed biological tissue. An acoustic response of the contrast agent is a function of a melting state of the lipid material.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for detecting a temperature characteristic with medical diagnostic ultrasound; and

FIG. 2 is a flow chart diagram of one embodiment of a method for detecting a temperature characteristic with medical diagnostic ultrasound.

### DETAILED DESCRIPTION OF THE DRAWINGS AND PRESENTLY PREFERRED EMBODIMENTS

A microbubble construction for contrast agents is modified to be sensitive to temperature. The temperature sensitivity is reflected in an amount of force used to destroy the contrast agents or cause the contrast agents to be absorbed. Response to ultrasound energy may indicate temperature sensitivity without destroying the contrast agents in other embodiments.

Ultrasound imaging of contrast agents is minimally invasive. The contrast agents are injected into the patient. Non-invasive ultrasound imaging then determines temperature characteristics. For example, steadily increasing mechanical index transmissions cause destruction of the contrast agents at different times. Contrast agents associated with higher temperatures are more likely to be destroyed than contrast agents associated with lower temperatures. The surviving contrast agent intensity and/or density is inversely related to temperature. The lowest density or intensity corresponds to the highest temperature for each transmission. Increasing the mechanical index generates a contour map or image of temperatures. The images formed from the transmissions indicate the temperature for different spatial locations.

The contrast agents are formed as microbubbles. The microbubbles are formed by application of ultrasound in a saline solution, centrifuging or other now known or later developed techniques. The microbubbles are of different sizes, wall thicknesses or shapes. Filtering or other techniques may be used to control the range of sizes. The microbubbles may be spherical with or without hollow insides. Microbubbles may be formed as plates or other structures.

The microbubbles are created for in vivo imaging with ultrasound. Due to the size, wall thickness, shape and material characteristics, the microbubbles are destroyable or absorbable in response to different levels of acoustic energy. Acoustic energy may cause the microbubbles to burst or rupture. One possible mechanism is a build-up of energy over a plurality of cycles of an acoustic waveform. The amount of build-up until rupture is based on the ratio of energy stored by the microbubble to the energy dissipated per cycle (Q). Other possible mechanisms may be more or less instantaneous, such as bursting in response to an increase in size or deformation of a relatively rigid wall in response to acoustic energy. Increased energy may result in the microbubble being more rapidly absorbed into fluids or deteriorated by chemical reaction. Regardless of the mechanism, thermal energy may cause the response of the microbubble to the acoustic energy to change. This change is detectable.

The material or structure determines the temperature dependence. Any material with a characteristic that is responsive to acoustic energy as a function of temperature may be used. In one embodiment, a lipid material is used. For example, the contrast agents include an organic monoacid with a 10, 11 or 12 carbon chain length. In one embodiment, the material is purified using zone refining, a molecular sieve or other technique to have a substantially same molecular weight and stereochemistry. A single type or a blended composition is used. Unbranched decanoic or short-branched (e.g., methyldecanoic or methylundecanoic) acids are used. Other chain lengths may be used. Other pure or non-pure saturated alkyl or other fatty acids may be used. Other materials than fatty acids may be used, such as a plastic, air filled Mylar, or albumin material.

The material and material characteristics determine a melting characteristic as a function of temperature. The microbubble melts somewhere within a range of temperatures, from high temperatures associated with inflammation of biological tissue to lower temperatures associated with non-inflamed biological tissue. The typical range of human temperatures is from 37 degrees Celsius of a normal tissue to 38 or 39 degrees of an inflamed tissue. Other ranges, such as broader (e.g., 35-41 degrees Celsius) or narrower, may be used. For example, lipids with a 10-12 carbon length have melting temperatures within 35-41 or 37-39 degrees Celsius. Other temperature ranges and/or associated temperature ranges may be used, such as associated with different contrast agents for patients with different basal temperatures or associated with non-human biological tissue (e.g., dogs or mice). Some of the contrast agents may have melting characteristics outside of the desired range.

The melting characteristic corresponds to a phase change. For example, at a temperature within the range, the contrast agent transitions from a solid (e.g., liquid crystal) to a fused (i.e . . . , liquid) state. The transition may occur outside the desired temperature range where the transition is gradual. The transition may alter the acoustic characteristic of the contrast agents without a full transition from a solid to a fused state. In other embodiments, the transition is sharp, such as associated with occurring over a 0.1 degree Celsius range. For example, a pure lipid may have a sharp melting characteristic. Different microbubbles through natural randomization or purposeful design may melt at different temperatures and/or with different melting transition rates. In order to accommodate natural individual and diurnal body-temperature variations, the transition temperatures within a collection of microspheres covers at least the desired range due to gradual transition and/or multiple sharp transition temperatures. This increases the chances that one patient will not melt all of the contrast agents, nor leave them all unmelted.

The mechanical properties of the contrast agent change over a narrow temperature range from a mechanically stronger solid state to a weaker fused state. The rarefaction or compression force required to destroy the contrast agent is less for the fused state. The acoustic response of the contrast agent is a function of a melting state of the lipid material. Contrast agents are more likely to be destroyed or absorbed in a range of temperatures associated with inflammation than associated non-inflamed biological tissue due to the state change caused by the temperature.

In additional or alternative embodiments, the structure of the microbubbles provides the temperature based response. For example, the microbubble includes different layers or combinations of materials. Each material reacts to temperatures in a desired range differently. The difference in reaction may strengthen or weaken the microbubbles, reducing or increasing the likelihood of destruction at particular temperatures. Other differences in reaction may be used, such as a different harmonic or other spectral response.

In another embodiment, the contrast agent responds to stimuli other than temperature. The response of the contrast agent is different for different chemical environments, different genes, different molecules, or other differences associated with patient tissue. For example, the contrast agent incorporates a gene or chemical marker. In response to binding, the structure of the contrast agent changes. The different stimuli cause the contrast agent to become weaker or stronger or to have a different reaction to acoustic energy.

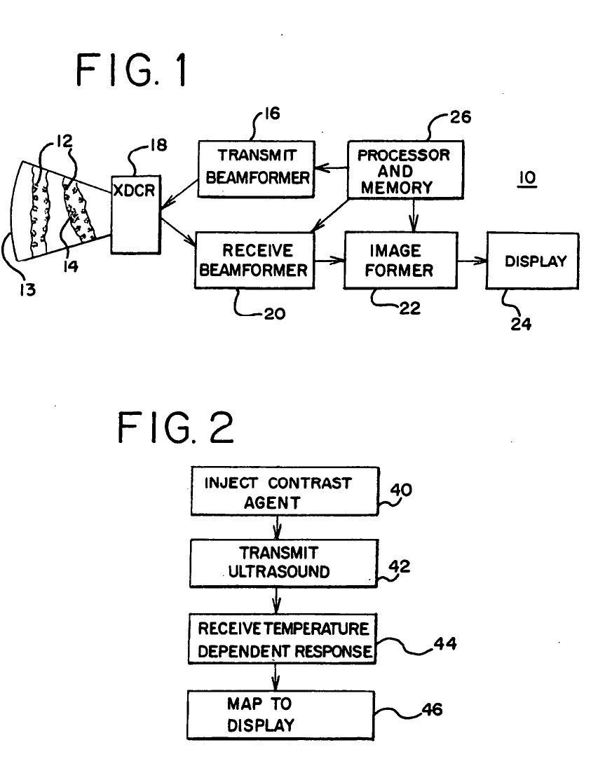

FIG. 1 shows one embodiment of a system 10 for applying acoustic energy to contrast agents 12 within a scan region 13 to detect inflammation 14. In the example of FIG. 1, the contrast agents 12 are within vessels or arteries, and the inflammation 14 is due to plaque being attacked by the patient's immune system. Other sources of inflammation or types of tissue are possible.

The system 10 includes a transmit beamformer 16, a transducer 18, a receive beamformer 20, an image former 22, a display 24, and a processor and memory 26. Additional, different or fewer components may be provided. In one embodiment, the system 10 is a medical diagnostic ultrasound imaging system. In other embodiments, the system 10 is an ultrasound therapy system. The system 10 may be or also include a workstation, such as a PACs workstation.

The transmit beamformer 16 includes one or more waveform generators, memories, pulsers, high voltage switches, phase rotators, delays, amplifiers, digital circuits, analog circuits, combinations thereof or other now known or later developed transmit beamformer components. In one embodiment, the transmit beamformer 16 is a programmable waveform beamformer, such as disclosed in U.S. Pat. No. 5,856,955 or 5,675,554, the disclosures of which are incorporated herein by reference. Sinusoidal, square wave, unipolar, or bipolar with any desired envelope may be generated using samples from a memory and a digital-to-analog converter. In another embodiment, the transmit beamformer 16 is a programmable waveform beamformer for coded excitations, such as disclosed in U.S. Pat. No. 6,213,947 or 6,241,674. In another embodiment, the transmit beamformer 16 is a pulser or switch based beamformer for generating unipolar or bipolar square waves. Amplifier or voltage source connections provide for different amplitudes. Switch frequency provides waveform frequency. Other now known or later developed beamformers may be used.

The transmit beamformer 16 generates waveforms in different channels. The waveforms are coded or not coded. The transmit beamformer 16 applies delay and apodization profiles to waveforms generated in different channels. The profiles focus the responsive acoustic energy along one or more scan lines in a given transmission. Alternatively, a plane, diverging, unfocused, or defocused wavefront is generated. The wavefront may be steered or unsteered. A single channel may be used.

The transducer 18 includes one or more elements. The elements are arrayed as a one dimensional, multi-dimensional (1.25, 1.5, 1.75, 2D), annular or other distribution. The elements are piezoelectric or capacitive membrane based elements.

In response to electrical energy from the transmit beamformer 16, the elements of the transducer 18 generate acoustic waveforms. The acoustic waveforms insonify the scan line through the region 12. The region 12 includes contrast agents 12. Any scan pattern may be used. By generating waveforms or sequences of transmissions for different scans with progressively increasing amplitude and/or decreasing frequency, the transmit beamformer 16 and transducer 18 may selectively destroy contrast agents. By changing amplitude or frequency as a function of time, contrast agents in different locations associated with different temperatures may be destroyed at different times. The lower amplitude or higher frequency energy may destroy some contrast agents and not others. The shift in amplitude or frequency may destroy other contrast agents after previous destruction events.

The transducer 18 receives acoustic echoes responsive to the transmissions. For example, echoes responsive to every transmission are received. As another example, echoes responsive to some of the transmissions are not used, but other echoes are received. For receiving acoustic echoes, the acoustic energy is converted to electrical signals by each element of a receive aperture.

The receive beamformer 20 includes one or more amplifiers, delays, phase rotators, filters, summers, mixers, demodulators, analog-to-digital converters, digital circuits, analog circuits, combinations thereof or other now known or later developed receive beamformer components. In one embodiment, the receive beamformer 20 is a receive beamformer disclosed in U.S. Pat. Nos. 5,685,308, 5,882,307 or 5,827,188, the disclosures of which are incorporated herein by reference. In another embodiment, the receive beamformer 20 includes a decoding receiver for applying a pulse compression function corresponding to a transmit code, such as disclosed in U.S. Pat. No. 6,213,947 or 6,241,674, the disclosures of which are incorporated herein by reference.

Receive beamformers 16 for receiving fundamental or harmonic information may be used. For example, the receive beamformer 20 includes a high pass or band pass filter for receiving at a second harmonic of a fundamental transmit frequency. Information at a fundamental or transmit frequency band may also or alternatively be used. As another example, the receive beamformer 20 includes a filter, multipliers, summer or subtractors for combining data responsive to different transmit waveforms to isolate information with a desired characteristic, such as even-harmonic information. Receive beamformers 16 for receiving echoes responsive to planar, broad or diverging wavefronts may be used, such as parallel receive beamformers or Fourier transform processors. Other receive beamformers or no receive beamformer may be used.

In one embodiment, the transmit beamformer 16 and receive beamformer 20 implement loss-of-correlation detection to detect contrast agent destruction. For example, any of the detectors and associated transmit and receive sequences disclosed in U.S. Pat. Nos. 6,494,841 and 6,682,482, the disclosures of which are incorporated herein by reference, are used. These detectors detect contrast agent information in response to different interpulse phase and/or amplitude modulation. Such detection methods may provide signals representing primarily contrast agent or contrast agent absent tissue information. In other embodiments, both contrast agents and tissue information are detected, such as with single pulse or multi-pulse harmonic or fundamental B-mode imaging. High power transmissions, low power transmissions or combinations of both may avoid or cause destruction of contrast agents as part of imaging contrast agents.

The image former 22 is a detector and scan converter. The image former 22 receives beamformed signals and outputs data for an image. The image is displayed on the display 24. The image former 22 may form one, two or three-dimensional images or representations.

The processor and memory 26 include a control processor, general processor, application specific integrated circuit, field programmable gate array, digital signal processor, digital circuit, analog circuit, combinations thereof or other now known or later developed processor. The memory is a cache, buffer, look-up table, RAM, ROM, database, removable media, optical, magnetic, combinations thereof or other now known or later developed memory. The processor and memory 26 may be a network of components, such as different processors for performing different operations in parallel or sequence.

In one embodiment, the processor and memory 26 receive the detected ultrasound information. The contrast agent information, such as detected reflected acoustic intensities or detected destruction, is mapped to colors or gray scale representing different temperatures. Locations associated with destruction of contrast agents in response to the different transmit amplitudes and/or frequencies indicate different temperatures. The relative or absolute temperatures are mapped. Different maps may be used for different depths. For example, the maps account for depth-dependent attenuation of the acoustic energy and the associated difference in energy applied to contrast agents. Gain adjustment may also be used. Alternatively, the image information from the different transmissions is shown sequentially to highlight change in destruction over time. Side-by-side presentation may also be used. In another alternative, a graphic overlay, such as contour lines, highlighting, numbers, text or other information indicating relative temperature is generated and overlaid on an image of the display 24 or displayed without an image.

The processor and memory 26 control the operation of the system 10. For example, the memory is a computer readable storage medium having stored therein data representing instructions executable by the programmed processor for detecting a temperature characteristic with a medical diagnostic ultrasound system. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

In one example embodiment, the instructions include controlling the transmit beamformer 16 to transmit, sequentially, acoustic energy at different intensities into a region. The receive beamformer 20 is controlled to receive signals responsive to the acoustic energy and contrast agents operable to change state as a function of temperature. The instructions cause one, two or three-dimensional scanning of a region. The received signals are for the region. The signals are responsive to contrast agents in intensity or by loss of correlation. Destruction or other changes in contrast agent are detected by loss of correlation, by identification of regions associated with threshold amounts of intensity, or other technique. The received signals are responsive to any contrast agents. The signals may be responsive to temperature due to a phase change from a solid state to a fused state as a function of temperatures associated with tissue. The signals are a function of the relative temperatures.

Spatial locations associated with destruction of the contrast agents are determined in response to the increasing amplitude and/or decreasing frequency of the waveforms in sequential transmissions. The change of state more likely results in destruction of the contrast agents in response to the acoustic energy. Contrast agents associated with increased temperature are more likely to be destroyed at lower amplitudes or higher frequencies, as compared to contrast agents associated with the lower, basal temperature. The relative temperature is mapped or otherwise displayed as a function of spatial location within the region.

The instructions are for the system 10 of FIG. 1 or a different system. Other embodiments, such as embodiments discussed herein, may be implemented by the instructions.

FIG. 2 shows a method for detecting a temperature characteristic with a medical diagnostic ultrasound system. The system is the system 10 of FIG. 1 or a different system. The acts of FIG. 2 are implemented in the order shown or a different order. Additional, different or fewer acts may be provided. For example, an image is generated from the temperature-dependent response without mapping in act 46. Sequential viewing, side-by-side viewing, or a combination of images from sequential scans may show areas of destruction or progressively fewer contrast agents, indicating areas of higher temperature.

In act 40, contrast agents are injected into the patient. A bolus of contrast agents is introduced into the blood stream of the patient. The bolus travels through the circulatory system to an artery or vessel of interest. In alternative embodiments, the contrast agents are introduced continuously, or through a catheter or in another now known or later developed method.

The contrast agents have desired melting characteristics appropriate for most or all patients. Alternatively, contrast agents are selected for a basal temperature of the patient. In other embodiments, the basal temperature of the patient is altered to correspond to the contrast agents.

The contrast agents have a characteristic which changes as a function of temperature. For example, the contrast agents have a melting characteristic at a temperature associated with inflammation or other disease state.

In act 42, ultrasound energy is transmitted. The transmission is along one or more scan lines, but a planar or diverging wavefront may be used. A plurality of microbubbles along the scan line or lines is insonified. Different transmissions are used to insonify different scan lines or groups of scan lines. Any scan pattern may be used to scan a two- or three-dimensional region having at least some microbubbles.

The region is a portion of a scan line, a two-dimensional region or a three-dimensional region. In one embodiment, the region includes a portion of a circulatory system, such as a vessel, artery, or the heart. In alternative embodiments, the region includes an organ or tissue of interest. The region may or may not include portions associated with increased temperature, such as inflamed tissue.

Some of the contrast agents will be adjacent to or contact the tissues within the region or fluids adjacent the tissues. As the contrast agents flow through the circulatory system or perfuse within tissue, the contrast agents may change based on the temperature.

In one embodiment, the microbubbles bind to tissue of interest. For example, virus vectors or ligands selectively bind to tissue of interest. As another example, acoustic energy is use to position the contrast agents adjacent to tissue or bind with tissue. For example, the methods, acts, instructions or systems disclosed in U.S. Pat. No. ______ (Publication No. ______ (Ser. No. 11/197,954 (Attorney Ref. No. 2005P09935US))), the disclosure of which is incorporated herein by reference, is used. In general, contrast agents are manipulated with acoustic radiation force while ultrasound imaging. Continuous waves for acoustic radiation force are transmitted. Substantially simultaneously, pulsed waves for imaging and/or contrast agent destruction are transmitted. Low mechanical index continuous and pulsed waves may be used to position contrast agents adjacent tissue. The acoustic radiation force may be transmitted with an amplitude profile and/or unfocused or defocused to minimize the effect of the continuous waves on imaging with the pulsed waves.

Alternatively or additionally, contrast agents bound or perfused within tissue are distinguished from free-flowing or moving contrast agents. For example, the methods, acts, instructions or systems disclosed in U.S. Pat. No. ______ (Publication No. ______ (Ser. No. 11/237,221 (Attorney Ref. No. 2005P13753US))), the disclosure of which is incorporated herein by reference, is used. Contrast agents are characterized with ultrasound. Flowing or unbound contrast agents are distinguished automatically from bound or relatively stationary contrast agents. The bound or relatively stationary contrast agents are highlighted on a display or used for relative or absolute temperature determination. A processor distinguishes different types of contrast agents or contrast agents in different binding states with relative signal strength or velocity. Attached contrast agents are differentiated from phagocytosed contrast agents. Monitoring absolute signal strength as a function of time may indicate binding.

In alternative embodiments, flowing contrast agents are used regardless of bonding state. Even without any bonding, the contrast agents may indicate relative or absolute temperature of fluid. The temperature of the fluid may indicate the temperature of adjacent tissue.

The scan of the region is repeated. The same or different scan format, focal positions, or scan lines are used for each sequential scan. The scan is repeated one or more times. Each repetition has a different intensity (e.g., amplitude) and/or frequency. For example, the density of scan lines is increased, increasing the intensity of acoustic energy applied to a given spatial location and corresponding contrast agents. In another example, a pulse repetition frequency is increased. As another example, the amplitude of the transmitted acoustic wavefront is increased. In another example, a frequency of the acoustic waveform is decreased. Combinations of waveform frequency, scan line density, pulse repetition frequency or amplitude may be used.

In one embodiment, each repetition has an increased mechanical index of a previous scan. The mechanical index is increased linearly or non-linearly in any step size. In general, the initial mechanical index is set at or below a level associated with destruction of contrast agents in a fused state. Higher initial settings may be used. Over two, three, four or more repetitions, the mechanical index is increased to a level associated with destruction of contrast agents in a solid state.

The mechanical index may be depth dependent. Due to depth dependent attenuation and/or any intervening contrast agent, the intensity of the transmitted acoustic energy may be less for greater depths. The mechanical index for a scanned field may be set or adjusted to provide a desired mechanical index or acoustic energy at a desired location. A greater range of mechanical index, a greater starting mechanical index, or other setting for acoustic intensity may be altered as function of depth of interest.

In act 44, a temperature dependent response is received. Acoustic reflections associated with contrast agents are received. The received signals correspond to no contrast agent, contrast agent or destruction of contrast agent. Correlation of received signals may indicate destruction of contrast agents. Relative intensity may also indicate destruction of some contrast agents. Other characteristics of the acoustic response of contrast agents may be used.

The temperature characteristic along at least a portion of the scan line is determined as a function of the response to the insonifying. Temperature characteristics are determined for one, two or three-dimensional regions, such as for one or more vessels.

Since contrast agents associated with higher temperature are more easily destroyed, the intensity of the acoustic response to contrast agents will be less for higher temperature regions. Alternatively or additionally, loss-of-correlation or other techniques may identify contrast agent destruction. Surviving microbubble density, shown by the intensity of the response or locations without loss of correlation, indicates or correlates with temperature.

Increasing the transmitted intensity differentiates different relative temperatures. Using a single transmit intensity indicates temperatures above and below a temperature at a given depth. Increasing the transmitted intensity sequentially delineates additional temperatures. The contrast agents are more easily destroyed as temperature increases. The response to increasing transmit intensity sequentially destroys remaining warmer, weaker contrast agents.

Using the response or destruction of contrast agents, different relative temperatures associated with different locations are determined. The relative temperature characteristic of the tissue for the scanned portion may aid in diagnosis or identification of inflammation. Absolute temperatures may be derived, such as from basal temperature, depth, original contrast agent density and transmit intensity. Alternatively, relative temperatures are used without conversion to absolute temperature.

Where temperature is to be measured at different depths, the relative temperature information may be adjusted. Due to depth dependent attenuation, contrast agents associated with a same temperature may be destroyed in response to different transmitted intensities. The mapping or intensity associated with detected destruction is altered to account for the depth dependent attenuation. For example, a color map relates a same color to different transmit intensities at different depths.

In act 46, the relative temperatures are mapped to display values. Gray scale or color values indicate different relative temperatures. The relative temperatures or values are scaled or not scaled as a function of depth. For example, two vessels generally parallel to the transducer at different depths are scanned after injection with contrast agents. One range of temperatures is displayed for the closer vessel, and another range of temperatures is displayed for the farther vessel. The temperatures are relative rather than absolute. Locations of inflammation in either of the vessels are determined by comparison to displayed values at similar depths. A different color or grayscale value may be displayed for a same temperature in each of the vessels. As another example, the mapping accounts for depth attenuation, so substantially the same color or display value is provided at different depths for a same absolute or relative temperature.

The mapped values are displayed as images or overlaid on an image, such as a B-mode or color flow image. The image indicates locations of different temperatures. Another display is a B-mode, color flow or other contrast agent image. By displaying multiple images in sequence or at a same time, the locations associated with the destruction or other acoustic response are perceived by comparison. Multiple images may be averaged or combined. The combination may provide different intensity levels for different locations. Locations with contrast agent and associated with less destruction have a higher combined intensity, indicating lower temperature. Other displays may be used, such as subtraction of images to identify changes in intensity as a function of increasing transmit intensities.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for detecting a temperature characteristic with a medical diagnostic ultrasound system, the method comprising:
insonifying a plurality of microbubbles along at least a scan line; and
determining the temperature characteristic along at least a portion of the scan line as a function of a response to the insonifying.

2. The method of claim 1 wherein the plurality of microbubbles are distributed over an at least two-dimensional region, wherein insonifying comprises insonifying over the at least two-dimensional region, and wherein determining comprises determining the temperature characteristic for different locations within the at least two-dimensional region.

3. The method of claim 2 further comprising generating a display of the temperature characteristic as a function of the different locations.

4. The method of claim 1 wherein insonifying comprises insonifying the plurality of microbubbles within a portion of a circulatory system, and wherein determining comprises determining the temperature characteristic of a the portion.

5. The method of claim 1 wherein insonifying comprises insonifying the plurality of microbubbles substantially bound to tissue.

6. The method of claim 1 wherein insonifying comprises insonifying sequentially with increasing mechanical index, and wherein determining comprises detecting destruction of the microbubbles as a function of the increasing mechanical index.

7. The method of claim 6 wherein determining comprises correlating surviving microbubble density with temperature.

8. The method of claim 1 wherein insonifying comprises insonifying the microbubbles, the microbubbles comprise lipid-based microbubbles having a phase change at a temperature of about 35-41 degrees Celsius.

9. The method of claim 8 wherein the microbubbles comprise lipid material having the phase change at a temperature of about 37-39 degrees Celsius.

10. The method of claim 1 wherein insonifying comprises insonifying the microbubbles, the microbubbles comprising material operable to more likely be destroyed or absorbed in a range of temperatures associated with inflammation than associated non-inflamed biological tissue.

11. In a computer readable storage medium having stored therein data representing instructions executable by a programmed processor for detecting a temperature characteristic with a medical diagnostic ultrasound system, the storage medium comprising instructions for:
transmitting, sequentially, acoustic energy at different intensities into a region;
receiving signals responsive to the acoustic energy and contrast agents operable to change state as a function of temperature, the signals associated with the region; and
determining a relative temperature as a function of the signals.

12. The instructions of claim 11 wherein determining comprises determining spatial locations associated with destruction of the contrast agents, the change of state more likely resulting in destruction of the contrast agents in response to the acoustic energy, contrast agents associated with increased temperature more likely to be destroyed at lower ones of the different intensities.

13. The instructions of claim 11 further comprising mapping the relative temperature as a function of spatial location within the region.

14. The instructions of claim 11 wherein receiving comprises receiving the signals responsive to contrast agents operable to change from a solid state to a fused state as a function of temperatures associated with tissue.

15. In contrast agents comprising microbubbles for in vivo imaging with ultrasound, the contrast agents destroyable or absorbable in response to different levels of acoustic energy, an improvement comprising:
a lipid material having a melting characteristic within a range of temperatures from temperatures associated with inflammation of biological tissue to temperatures associated with non-inflamed biological tissue, an acoustic response of the contrast agent being a function of a melting state of the lipid material.

16. The improvement of claim 15 wherein the acoustic response corresponds with the contrast agents being more likely to be destroyed or absorbed in a range of temperatures associated with inflammation than associated non-inflamed biological tissue.

17. The improvement of claim 15 wherein the range of temperatures comprises 37-39 degrees Celsius.

18. The improvement of claim 15 wherein the lipid material comprises an organic monoacid with a 10 to 12 carbon chain length.

19. The improvement of claim 15 wherein the melting characteristic comprises a phase change from solid or liquid-crystal to fused.
